(19) United States
(12) Patent Application Publication
(10) Pub. No.: US 2023/0405823 A1
(43) Pub. Date: Dec. 21, 2023

(54) MONITORING METHOD AND ROBOTIC SYSTEM

(71) Applicant: Siemens Healthcare GmbH, Erlangen (DE)

(72) Inventors: Hayo Knoop, Forchheim (DE); Elisabeth Preuhs, Erlangen (DE); Markus Kowarschik, Nürnberg (DE); Stephan Kellnberger, Erlangen (DE)

(21) Appl. No.: 18/210,051

(22) Filed: Jun. 14, 2023

(30) Foreign Application Priority Data
Jun. 15, 2022 (DE) 10 2022 206 067.9

(51) Int. Cl. B25J 9/16; B25J 19/02; A61B 34/30; A61B 34/00

(52) U.S. Cl. CPC B25J 9/1674; B25J 9/1679; B25J 19/026; A61B 34/30; A61B 34/25

(57) ABSTRACT

For a simple monitoring of a robotic system that is configured for robot-assisted actuation of a movement of a medical object in a hollow organ of a patient, the robotic system includes at least one drive system, a robot control unit, and an acoustic sensor. A method is provided and includes receiving, by the acoustic sensor, acoustic signals of the robotic system during the operation of the robotic system for moving the medical object. At least one signal pattern is recognized in the received acoustic signals. The at least one recognized signal pattern is evaluated with respect to an associated action flow of at least one component of the robotic system. The method includes checking whether the action flow is an intended action flow, and actuating an action if the action flow is unintended.

This application claims the benefit of German Patent Application No. DE 10 2022 206 067.9, filed on Jun. 15, 2022, which is hereby incorporated by reference in its entirety.

### BACKGROUND

The present embodiments relate to monitoring a robotic system.

Robotic systems having a drive mechanism (e.g., robotic manipulators) use electric drives in order to move medical objects such as catheters or guide wires similar to by a human hand through hollow organs of the human body (e.g., within the scope of endovascular procedures). For this purpose, the drives are mechanically connected to the respective medical object. During the manipulation, attempts are made to perform translation movements and rotations using the medical objects. In so doing, undesired slipping (e.g., through) of the medical objects, also referred to as ‘slip,’ frequently occurs. This behavior leads to multiple problems: the medical objects are not moved in a manner that is intended and planned by the operator activating the electric drive. A display of translations and rotations that are calculated by evaluations of the drives is incorrect due to the slip. As a result, it is possible, for example, for measurements that are likewise performed by evaluations of the drives to be incorrect and imprecise.

Although mechanical elements that are arranged on the medical object in order to prevent slip do exist, the mechanical elements are frequently counter-productive for the mechanical loadability of the medical objects that are originally designed for use by the human hand. Further, it is possible to perform a visual check using X-ray fluoroscopy. However, this is only performed in 2D or 2×2D owing to the high radiation exposure and is therefore only an estimation in the case of a three dimensional passage through a vessel. Within the scope of this check, an activation of the electric drive with the current X-ray image of the catheter or guide wire by the human is compared with the displayed values and measurements.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method for robotic systems that are used for robot-assisted actuation of a movement of a medical object in a hollow organ of a patient, that allows low-radiation and effective monitoring of a slip is provided. As another example, a robotic system that is suitable for performing the method is provided.

The method in accordance with the present embodiments for monitoring a robotic system that is configured for robot-assisted actuation of a movement of a medical object in a hollow organ of a patient is provided. The robotic system includes at least one drive system, a robot control unit, and an acoustic sensor. The method includes receiving, by the at least one acoustic sensor, acoustic signals of the robotic system (e.g., from at least one drive of the drive system) during the operation of the robotic system for moving the medical object. The method includes recognizing at least one signal pattern in the received acoustic signals, evaluating the at least one recognized signal pattern with respect to an associated action flow of at least one component of the robotic system, checking whether the action flow is an intended action flow, and actuating an action if the action flow is unintended. For example, an evaluation is performed with respect to a slip (e.g., an unintended slip; slipping through) between a drive and a medical object, and if a slip is determined, then an action is actuated since the slip is fundamentally unintended.

The method renders it possible to check in a simple manner, effectively, and without exposing the patient to X-ray radiation whether the movement of the medical object is performed by the robotic system as intended or whether deviations, interferences, or errors, such as, for example, slip, occur. This may be performed without outlay merely by evaluating acoustic recordings, for example, of noises of the drive. The present embodiments are based on the knowledge that incorrect functions, such as, for example, the slipping through/slip generate a characteristic noise that differs from a continuous movement and clearly indicates a corresponding incorrect function. Slipping through generates a re-occurring signal pattern, for example, by exceeding a resistance. If such a signal pattern is recognized and identified, an appropriate action is performed. The action may either be performed after or even also during the incorrect function if, for example, it is determined very quickly at the beginning that an incorrect function has occurred. Further, the method renders it possible to correct erroneous measurement values of the robotic system that occur as a result of the incorrect function, such as, for example, the slipping through/slip.

According to one embodiment, the actuated action includes outputting a message, outputting a warning, outputting an action proposal, automatically interrupting the operation of the robotic system, and/or an automatic corrective action so as to eliminate or mitigate the unintended action flow. The message may be output (e.g., within the scope of a display on a screen, a warning may be provided in a visual, acoustic, or haptic manner). An action proposal may also be displayed, for example, on a screen or may be output by an acoustic announcement (e.g., a request to adjust the current movement). The action may also be performed in dependence upon the type of the unintended action flow. Thus, the operation may be interrupted, for example, in the case of a dangerous incorrect function. A corrective action may be performed automatically or semi-automatically by the robotic system. For example, in the case of a slip, the corrective action may include a number of short forward and backward movements or an activation or action amplification of a mechanical component in the drive in order to mitigate and/or prevent the occurring slip. Such actions render it possible to initially realize the negative effects of incorrect functions, such as, for example, slip, and subsequently to reduce or completely prevent them.

According to a further embodiment, in order to check whether the action flow is an intended action flow, a comparison is performed with a planning guideline and/or a database and/or a control guideline. The comparison renders it possible to clearly establish whether the current action flow was actually planned this way. If, for example, a slip occurs, then it is possible to deduce from the planning or control guidelines that this was unintended. In the case of some action flows, it is also possible to establish right from the beginning that the action flows may never belong to the intended action flows, whereas in the case of other action flows, a situation-related intention may be present.

According to a further embodiment, at least one pre-trained machine-learning algorithm is used for evaluating and checking the signal pattern. A machine-learning algorithm is based, for example, on neural networks; it is possible, for example, to use a Deep Learning algorithm. The use of machine-learning algorithms may be particularly advantageous if a quick and self-optimizing analysis is necessary. Repeated and varied model tests and comparisons with the actual geometry enable the machine-learning algorithm to be trained, for example, as a ground truth algorithm.

According to a further embodiment, a continuous monitoring of the robotic system is performed during the operation thereof, and the monitoring is terminated when the robotic system is deactivated. In this manner, continuous monitoring of the robotic system is provided with error minimization. For example, the method is automatically trigger started by an activation of the robotic system.

According to a further embodiment, in addition to the acoustic monitoring, a further monitoring method is used (e.g., monitoring using imaging). In this case, the movement of the medical object in the hollow organ of the patient may be observed and monitored (e.g., using fluoroscopy images). Additional monitoring renders it possible to almost exclude errors, and such movements may be performed and monitored for the patient.

The present embodiments include a robotic system that includes at least one robot control unit and a robot-assisted drive system having a drive and a drive mechanism. The drive system is configured, based on control signals of the robot control unit, to move a medical object in a hollow organ of a patient. The robotic system includes at least one acoustic sensor that is configured so as to receive acoustic signals of the robotic system, and is arranged on the robotic system. The robotic system includes an evaluating unit that is configured to recognize signal patterns and to evaluate the signal patterns with respect to an associated action flow of at least one component of the robotic system. In one embodiment, the robot control unit is configured to actuate an action (e.g., to output a message, to output a warning, to output an action proposal, to automatically interrupt the operation of the robotic system, and/or to automatically perform corrective action so as to eliminate the unintended action flow). The robotic system has, for example, a display unit for displaying messages and/or warnings and/or action proposals.

### BRIEF DESCRIPTION OF THE DRAWINGS

FIG. 1 shows a sequence of acts of one embodiment of a method for monitoring a robotic system;

FIG. 2 shows a view of one embodiment of a robotic system for performing the method; and

FIG. 3 shows a further view of a robotic system having a number of acoustic sensors.

### DETAILED DESCRIPTION

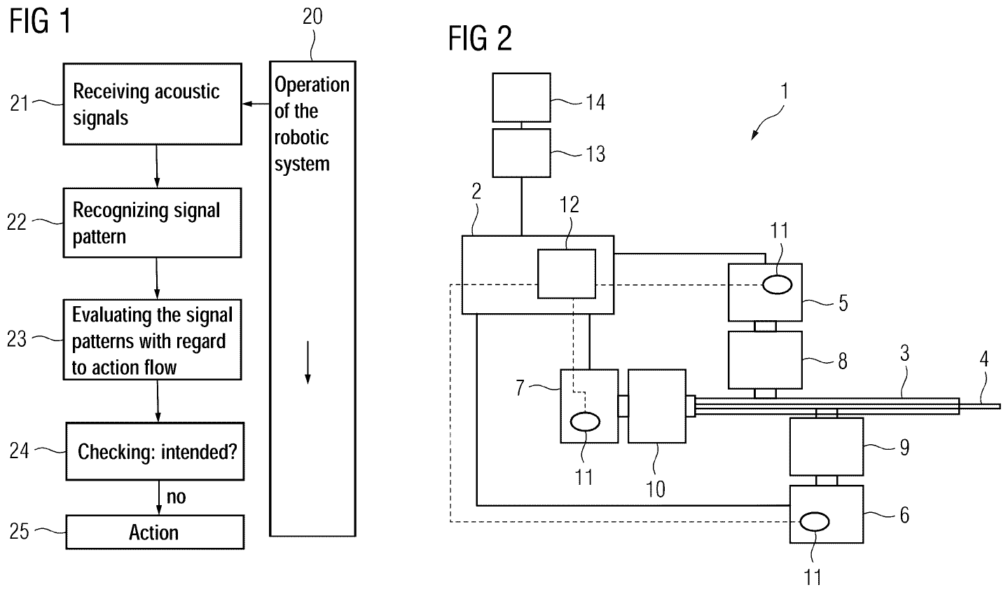

FIG. 1 illustrates a sequence of acts of one embodiment of a method for monitoring a robotic system that provide particularly simple, effective, and low-radiation monitoring of the function of the robotic system. The robotic system is used to navigate medical objects, such as, for example, guide wires, catheters, and other devices, semi-automatically or automatically through a hollow organ (e.g., a blood vessel) of a patient. FIG. 2 illustrates an example of a robotic system 1 having multiple drives 5, 6, 7 and a robot control unit 2.

During operation 20 of a robotic system 1 (e.g., during a robot-assisted navigation of a catheter 3 and a guide wire 4 through a hollow organ of a patient), acoustic signals from at least one component of the robotic system 1 are received in a first act 21 by at least one acoustic sensor 11 (e.g., a microphone). The component may be, for example, a drive 7 that drives a drive mechanism, such as, for example, a manipulator 10. The manipulator 10 is connected directly or indirectly to the medical object (e.g., the guide wire 4 or the catheter 3). The component may also be a different mechanical component, a component for transferring force to the medical object or the medical object itself, or two or more medical objects that are mutually connected or nested in one another (e.g., catheter or guide wire). It is also possible, using a number of acoustic sensors 11 (e.g., microphones), to receive acoustic signals from a number of components.

In a second act 22, the received acoustic signals are read with respect to signal patterns, or signal patterns are detected in the acoustic signals. This may be performed, for example, by software algorithms (e.g., also using artificial intelligence or pre-trained machine-learning algorithms, such as using deep neural networks). Signal patterns may be continuously recognized or read in this manner. In a third act 23, the recognized signal patterns are evaluated with respect to an associated action flow of at least one component of the robotic system. This may likewise be performed by software algorithms, also in this case, for example, using artificial intelligence or pre-trained machine-learning algorithms (e.g., using deep neural networks). It is possible to provide that in each case an acoustic sensor is assigned respectively to a predetermined component so that it is possible to limit the evaluation to this component. Thus, for example, in FIG. 2, an acoustic sensor 11 is assigned to a first drive 4, an acoustic sensor 11 is assigned to a second drive 6, and an acoustic sensor 11 is likewise assigned to a third drive 7. It is also possible to continuously evaluate the signal patterns.

An action flow that is associated with a signal pattern is understood in this case to be by which process and/or which function and/or which incorrect function and/or which event the corresponding characteristic signal pattern has been generated. The present embodiments are based on the knowledge that each action flow (e.g., each process and/or function and/or incorrect function and/or event) generates a characteristic noise (e.g., signal pattern) that differs from any other signal patterns of other action flows, and as a result, is clearly identifiable. Thus, for example, the signal pattern of the drive in the case of a continuous movement with translation or rotation of the guide wire differs from the signal pattern that a slipping through/slip of the guide wire generates, since in that case, a resistance is exceeded, for example. Such a signal pattern is clearly recognizable, and as a result, is easily identifiable in such an evaluation. Other processes, events, or functions may also be unambiguously assigned. An appropriate machine-learning algorithm may be trained prior to use for the method with the possibly occurring signal patterns. As a result, it is also possible to differentiate short pattern pulses from longer lasting patterns.

If the evaluation indicates that a predetermined action flow has occurred, a check is performed in a fourth act as to whether the action flow is an intended action flow (e.g., an action flow that is intended in the manner in which it has occurred). This may be performed, for example, with the aid of a comparison with a planning guideline, a database, or a control guideline. It is also possible to store which action flows are intended, and which are not, in a storage device or in the evaluation software itself. It is also possible, for example, to store that slip is unintended during a navigation procedure and a uniform movement of the medical object is intended.

If an unintended action flow is determined, an action is performed subsequently in a fifth act 25. If, for example, it is stored that a slip or an incorrect function is unintended, then, if a slip is determined in the case of a movement, an action is subsequently actuated in a fifth act 25. The actuated action may be, for example, outputting a message, outputting a warning, outputting an action proposal, automatically interrupting the operation of the robotic system, and/or an automatic corrective action so as to eliminate the unintended action flow.

A warning may be output in an acoustic, haptic, or visual manner, for example (e.g., as a colored light or as output text on a monitor). Additionally or alternatively, a request may be made for a user action that is a general action or an action that is adapted to the incorrect function (e.g., for cleaning or checking predetermined components or also for manually continuing the movement). Further, it is possible, for example, to suggest or automatically actuate additional movements of the components (e.g., short forward and backward movements) in order to eliminate the unintended action flow (e.g., slip). It is also possible, for example, to activate further or the same mechanical components or amplify their action in order to correct the unintended action flow. It is also possible to correct the target values originally calculated by activating the component (e.g., drive) and to activate a repeated (e.g., semi-automatic) movement. In critical situations, it is also possible to completely stop the operation of the robotic system. Further, the unintended action flow may be recorded. Thus, it is possible to subsequently correct erroneous measurement values of the robotic system that occur as a result of slipping through/slip, for example.

The machine-learning algorithm(s) that are used for the method (e.g., deep neural networks, long-short term memories, or recurrent neural networks) may be pre-trained using large amounts of data. For this purpose, automatic movements may be performed, for example, by an appropriate component (e.g., a drive mechanism, such as a manipulator, may perform a number of passes). Training may be performed by repeated and varied model tests and comparisons made with the actual geometry as ground truth.

The recognition of signal patterns and the evaluation thereof may be performed online or live in a very short time. By appropriately training the algorithm used, it is also possible to recognize parts of typical signal patterns at an early stage so that in many cases an occurring problem (e.g., unintended action flow) may be identified and counteracted at an early stage. In this manner, signal patterns may be used to predict the next movements and/or events (e.g., whether and when slip will occur). This information may then likewise be used to actuate actions (e.g., in order to output warnings or to perform appropriate automatic actions so as to prevent the event (slip)).

In addition to the acoustic monitoring by the acoustic sensors, it is also possible to use a further monitoring method in order to observe the robotic system or the movements of the medical object through the hollow organ of the patient. Thus, monitoring may be performed by fluoroscopy (e.g., X-ray imaging), for example. The corresponding images captured by an X-ray machine may also be evaluated (e.g., automatically by algorithms). The results may be coupled (e.g., temporally) with the results of the acoustic monitoring in order to obtain an even better evaluation of the navigation of the medical object and to be able to optimize the treatment of the patient. Fluoroscopy images may also be used for training the machine-learning algorithm for acoustic monitoring.

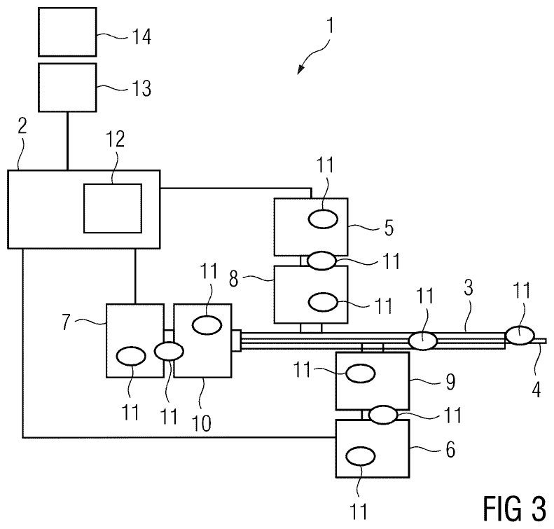

The exemplary robotic system 1 (see FIG. 2), in addition to the robot control unit 2 and multiple drives 5, 6, 7, also has multiple mechanical couplings 8, 9, and 10 that are associated with the respective drives 5, 6, and 7. The manipulated medical objects of the robotic system 1 are a catheter 3 and a guide wire 4 that may be moved in this manner through a hollow organ of a patient. An acoustic sensor 11 that receives acoustic signals of the acoustic sensors 11 is arranged in each case on the first drive 5, the second drive 6, and the third drive 7. The robotic control unit 2 controls, for example, in a semi-automatic manner by a user input using an input unit 13 or in a fully automatic manner, a movement of the catheter 3 and the guide wire 4 through the hollow organ. The received acoustic signals are evaluated by an evaluating unit 12 with regard to signal patterns and associated action flows. The evaluating unit 12 may perform this, for example, using machine-learning algorithms. Messages, warnings, or instructions may be output at a display unit 14. FIG. 3 illustrates a robotic system 1 similar to that in FIG. 2, but, however, with a number of acoustic sensors 11 at different positions on the robotic system 1. In addition to the drives, the acoustic sensors may also be attached, for example, at the connection to the medical object, at the mechanical couplings at their transitions, or at the medical object itself.

Typical robotic systems for which the method is applicable, provided the robotic systems are modified by acoustic sensors, an evaluation unit, and appropriate software, include the CorPath GRX system from Corindus, Inc. or an LBR system having a catheter. In the case of the former, the acoustic sensors may be arranged, for example, on the cassette of the drive unit.

The monitoring method uses acoustic sensors for receiving acoustic signals from components of a robotic system in order, by evaluating signal patterns, to render it possible to improve the robot-assisted movement of a medical object in a hollow organ of a patient or to optimize the navigation.

The present embodiments may be briefly summarized in the following manner. For a particularly simple monitoring of a robotic system that is configured for the robot-assisted actuation of a movement of a medical object in a hollow organ of a patient, the robotic system having at least one drive system, a robot control unit, and an acoustic sensor, a method that includes the following acts is provided: using the at least one acoustic sensor, receiving acoustic signals of the robotic system during the operation of the robotic system for moving the medical object; recognizing at least one signal pattern in the received acoustic signals; evaluating the at least one recognized signal pattern with respect to an associated action flow of at least one component of the robotic system; checking whether the action flow is an intended action flow; and actuating an action if the action flow is unintended.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for monitoring a robotic system that is configured for robot-assisted actuation of a movement of a medical object in a hollow organ of a patient, the robotic system including at least one drive system, a robot control unit, and an acoustic sensor, the method comprising:
receiving, by the acoustic sensor, acoustic signals of the robotic system during operation of the robotic system for moving the medical object;
recognizing at least one signal pattern in the received acoustic signals;
evaluating the at least one recognized signal pattern with respect to an associated action flow of at least one component of the robotic system;
checking whether the associated action flow is an intended action flow; and
actuating an action when the associated action flow is unintended.

2. The method of claim 1, wherein the actuated action includes outputting a message, outputting a warning, outputting an action proposal, automatically interrupting the operation of the robotic system, an automatic corrective action so as to eliminate the unintended action flow, or any combination thereof.

3. The method of claim 1, wherein checking whether the associated action flow is the intended action flow comprises comparing the associated action flow with a planning guideline, a database, a control guideline, or any combination thereof.

4. The method of claim 1, wherein the acoustic signals are received from at least one drive of the at least one drive system.

5. The method of claim 1, wherein at least one pre-trained machine-learning algorithm is used for evaluating and checking the signal pattern.

6. The method of claim 1, further comprising:
performing a continuous monitoring during the operation of the robotic system;
terminating the continuous monitoring on deactivation of the robotic system.

7. The method of claim 1, wherein the method is trigger started by an activation of the robotic system.

8. The method of claim 1, further comprising using a further monitoring method.

9. The method of claim 8, wherein the further monitoring method includes monitoring by imaging.

10. The method of claim 1, further comprising:
performing an evaluation with respect to an unintended slip between a drive and a medical object; and
actuating an action when the unintended slip is determined.

11. A robotic system comprising:
a robot control unit;
a robot-assisted drive system comprising:
a drive; and
a drive mechanism, wherein the drive system is configured to move a medical object in a hollow organ of a patient based on control signals of the robot control unit;
an acoustic sensor configured to receive acoustic signals of the robotic system, the acoustic sensor being arranged on the robotic system; and
an evaluating unit configured to:
recognize signal patterns; and
evaluate the signal patterns with respect to an associated action flow of at least one component of the robotic system.

12. The robotic system of claim 11, wherein the robot control unit is configured to actuate an action.

13. The robotic system of claim 12, the actuation of the action comprises:
output of a message;
output of a warning;
output of an action proposal;
automatic interruption of the operation of the robotic system;
performance of corrective action so as to eliminate the unintended action flow; or
any combination thereof.

14. The robotic system of claim 11, further comprising a display unit configured to display messages, warnings, action proposals, or any combination thereof.

15. The robotic system of claim 13, further comprising a display unit configured to display messages, warnings, action proposals, or any combination thereof.
